(19) United States
(12) Patent Application Publication
(10) Pub. No.: US 2023/0108062 A1
(43) Pub. Date: Apr. 6, 2023

(54) SENSOR MODULE FOR MEASURING BLOOD PRESSURE AND WRIST-WORN PORTABLE BLOOD PRESSURE MEASURING DEVICE

(71) Applicant: CHARMCARE CO., LTD., Seoul (KR)

(72) Inventor: Dong Hwa LEE, Yongin-si (KR)

(21) Appl. No.: 18/073,985

(22) Filed: Dec. 2, 2022

Related U.S. Application Data
(63) Continuation of application No. PCT/KR2021/006955, filed on Jun. 3, 2021.

(30) Foreign Application Priority Data
Jun. 3, 2020 (KR) 10-2020-0066843

(51) Int. Cl. A61B 5/021; A61B 5/00

(52) U.S. Cl. CPC A61B 5/02141; A61B 5/681; A61B 5/6844; A61B 2562/0247; A61B 2562/043; A61B 2562/0261

(57) ABSTRACT

Provided is a sensor module according to the present disclosure including a base portion, a first pressure sensor portion and a second pressure sensor portion coupled to the base portion and arranged adjacent to each other, and a hard structure layer coupled to an upper portion of the first pressure sensor portion. A step difference of a first distance is formed between an upper portion of the hard structure layer and the upper portion of the second pressure sensor portion.

### BACKGROUND

### 1. Field

The present disclosure relates to a blood pressure measuring device, and more particularly, to a sensor module for measuring a blood pressure and a wrist-wearable blood pressure measuring device using the same.

### 2. Description of the Related Art

Recently, interest in health care has been increased, and as the number of patients with a high blood pressure and a low blood pressure increases, research on a wearable device for measuring a blood pressure that may conveniently check their own blood pressure is being actively conducted.

Particularly, a wearable device for measuring a blood pressure by using an optical sensor and a pressure sensor to improve portability of a blood pressure measuring device is being developed.

A known wearable device for measuring a blood pressure by using a pressure sensor includes a wrist band including an air pump for checking a blood pressure in a state of being worn on the wrist. The known wearable device for measuring a blood pressure by using a pressure sensor measures the blood pressure by compressing the wrist by using the air pump of the wrist band, and accordingly, there is a problem in that a user is inconvenient during a compression process for measuring a blood pressure.

An example of the related art includes Korean Patent Publication No. 10-2016-0063471 (Title of the Invention: WRISTBAND TYPE BLOOD PRESSURE MEASURING APPARATUS).

### SUMMARY

According to a first aspect of the present disclosure, a sensor module for measuring a blood pressure includes a base portion, a first pressure sensor portion and a second pressure sensor portion coupled to the base portion and arranged adjacent to each other, and a hard structure layer coupled to an upper portion of the first pressure sensor portion. In this case, a step difference of a first distance is formed between an upper portion of the hard structure layer and the upper portion of the second pressure sensor portion.

According to the first aspect of the present disclosure, the sensor module may include a controller configured to control operations of the first pressure sensor portion and the second pressure sensor portion and configured to measure a blood pressure of a blood pressure measurement target blood vessel based on a first pressure detected by the first pressure sensor portion and a second pressure detected by the second pressure sensor portion. In this case, the controller may estimate a second distance indicating a distance from the upper portion of the hard structure layer to the blood pressure measurement target blood vessel based on the first pressure, the second pressure, and the first distance and may calculate a blood pressure of the blood pressure measurement target blood vessel based on a value obtained by multiplying the second distance by the first pressure or a value obtained by multiplying a sum of the first distance and the second distance by the second pressure.

According to a second aspect of the present disclosure, a wrist-worn portable blood pressure measuring device includes a main body unit including a display unit for displaying blood pressure information, a power supply unit, and a controller, a wrist strap coupled to the main body unit, and a sensor module coupled to one of the main body unit and the wrist strap to measure a blood pressure. In this case, the sensor module may include a first pressure sensor portion and a second pressure sensor portion which are coupled to a base portion and arranged adjacent to each other, and a hard structure layer coupled to an upper portion of the first pressure sensor portion, and a step difference of a first distance may be formed between an upper portion of the hard structure layer and an upper portion of the second pressure sensor portion.

According to the second aspect of the present disclosure, the controller of the wrist-worn portable blood pressure measuring device may estimate a second distance indicating a distance from the upper portion of the hard structure layer to a blood pressure measurement target blood vessel based on a first pressure, a second pressure, and the first distance and may calculate a blood pressure of the blood pressure measurement target blood vessel based on a value obtained by multiplying a second distance by the first pressure or a value obtained by multiplying a sum of the first distance and the second distance by the second pressure and may output the calculated blood pressure through the display unit. In this case, there is a step difference of a first distance between the upper portion of the hard structure layer coupled to the upper portion of the first pressure sensor portion and the upper portion of the second pressure sensor portion.

According to a third aspect of the present disclosure, a sensor module for measuring a pressure includes a first electrode including N separated electrode wires stacked in parallel to each other on an upper surface and a lower surface of a base layer and having inner bending repeated for each unit area, a second electrode which includes N separated electrode wires stacked in parallel to each other on an upper surface and a lower surface of a base layer and has inner bending repeated for each unit area and in which the electrode wires of the first electrode cross the electrode wires of the second electrode, and a plurality of dielectric layers inserted between the electrode wires of the first electrode and the electrode wires of the second electrode that arranged to face and cross each other. In this case, the sensor module further comprises a hard structure layer coupled to an upper surface of the sensor module to cover a predetermined area of the sensor module, and a step difference of a first distance may be formed between an upper portion of the hard structure layer and an upper portion of the sensor module not covered by the hard structure layer.

According to the second aspect of the present disclosure, the controller of the sensor module may estimate a second distance indicating a distance from an upper portion of the hard structure layer to the blood pressure measurement target blood vessel based on the first pressure, the second pressure, and a first distance and may calculate a blood pressure of the blood pressure measurement target blood vessel based on a value obtained by multiplying the second distance by the first pressure or a value obtained by multiplying a sum of the first distance and the second distance by the second pressure.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a block diagram illustrating a configuration of a sensor module for measuring a blood pressure according to an embodiment of the present disclosure;

FIGS. 2A to 2C illustrate a principle of a sensor module for measuring a blood pressure, according to an embodiment of the present disclosure;

FIGS. 3A to 3C are perspective views illustrating a configuration of a sensor module according to an embodiment of the present disclosure;

FIG. 4 is a cross-sectional view illustrating a configuration of a wrist-worn portable blood pressure measuring device according to an embodiment of the present disclosure;

FIG. 5 is a block diagram illustrating a configuration of a main body unit of the wrist-worn portable blood pressure measuring device according to an embodiment of the present disclosure;

FIG. 6 is a flowchart of a blood pressure measuring method using a pressure sensor, according to an embodiment of the present disclosure;

FIG. 7 is a graph illustrating a change in arterial blood pressure over time;

FIGS. 8 to 10 are views illustrating a stacked structure and detailed configuration of a sensor module for measuring a pressure according to an embodiment of the present disclosure;

FIG. 11 is a cross-sectional view taken along line D of FIG. 10;

FIGS. 12 to 13 are views illustrating that a capacitor is formed in a sensor module for measuring a pressure according to an embodiment of the present disclosure; and

FIGS. 14 and 15 are perspective views illustrating a configuration of the sensor module for measuring a pressure according to an embodiment of the present disclosure.

### DETAILED DESCRIPTION

Hereinafter, embodiments of the present disclosure will be described in detail with reference to the accompanying drawings such that those skilled in the art to which the present disclosure belongs may easily implement the present disclosure. However, the present disclosure may be embodied in various different forms and is not limited to the embodiments described herein. In addition, in order to clearly illustrate the present disclosure in the drawings, parts irrelevant to the descriptions are omitted, and similar reference numerals are attached to similar parts throughout the specification.

Throughout the specification, when a portion is “connected” or “coupled” to another portion, this includes not only a case of being “directly connected or coupled” but also a case of being “electrically connected” with another element interposed therebetween.

Throughout the specification, when a member is said to be located “on” another member, this includes not only a case in which the member is in contact with another member but also a case in which there is another member between the two members.

Hereinafter, an embodiment of the present disclosure will be described in detail with reference to the accompanying drawings.

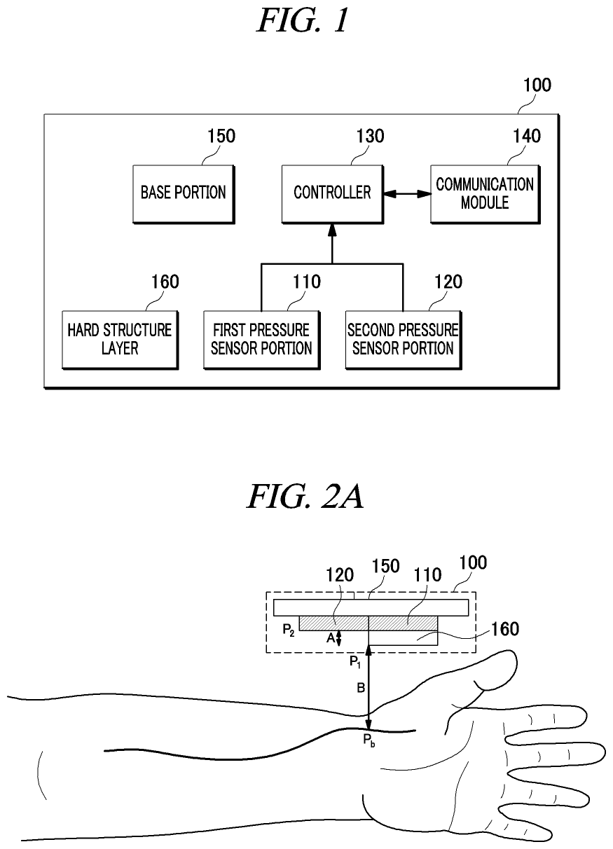

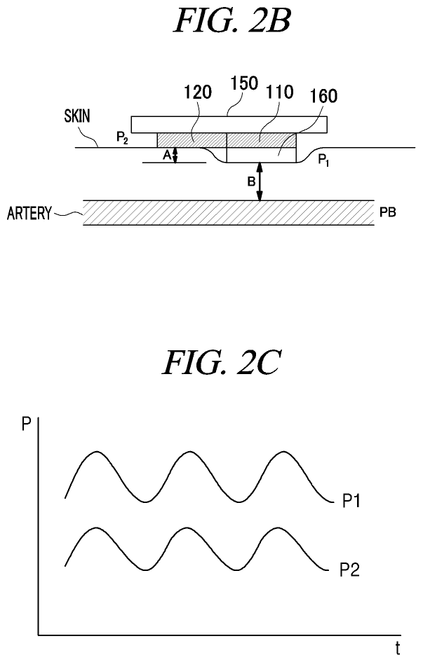

FIG. 1 is a block diagram illustrating a configuration of a sensor module for measuring a blood pressure according to an embodiment of the present disclosure, and FIG. 2 is a diagram illustrating a principle of the sensor module for measuring a blood pressure according to an embodiment of the present disclosure.

As illustrated, a sensor module 100 for measuring a blood pressure may include a first pressure sensor portion 110, a second pressure sensor portion 120, a hard structure layer 160, a controller 130, a communication module 140, and a base portion 150.

At this time, the first pressure sensor portion 110 and the second pressure sensor portion 120 may each include a strain gauge-based semiconductor thin film sensor, a capacitive thin film sensor for detecting capacity change according to a pressure, a piezoresistive sensor using a piezo resistance effect, or other various pressure sensors.

Here, the strain gauge-based thin film sensor has an advantage that only a resistance change needs to be controlled by arranging four resistors on a diaphragm in the form of a Wheatstone bridge. The capacitive thin film sensor is strong against heat resistance and corrosion resistance and has an advantage that a pressure is measurable very precisely. The piezoresistive sensor has advantages in that there are high sensitivity, high linearity, and high reproducibility when measuring a pressure and mass-production may be performed.

Referring to FIG. 2, the hard structure layer 160 may be coupled to an upper portion of the first pressure sensor portion 110, and thus, a step difference of a first distance may be formed between an upper portion of the hard structure layer 160 and an upper portion of the second pressure sensor portion 120.

The controller 130 may calculate a distance between the first pressure sensor portion 110 and an artery, and a blood pressure of the artery based on pressures measured by the first pressure sensor portion 110 and the second pressure sensor portion 120 and a difference in distance between the upper portion of the hard structure layer 160 and the upper portion of the second pressure sensor portion 120. The upper portions described above may indicate a 6 o'clock direction in FIGS. 2A and 2B.

The communication module 140 may transmit and receive data to and from a communication module 240 of a main body unit 200 and various external devices (a server and a terminal) in respectively set communication formats.

The first pressure sensor portion 110 and the second pressure sensor portion 120 are disposed adjacent to each other in the base portion 150. In addition, the controller 130 and the communication module 140 may be coupled to the base portion 150 or a back surface of the base portion 150, or may be included in a separate housing to which the base portion 150 is coupled.

The sensor module 100 may be used in combination with a smart watch, a wrist-worn portable blood pressure measuring device, and so on. In addition, a user may measure a blood pressure simply by attaching the sensor module to the wrist with a fixing means such as a tape, or by inserting the sensor module into clothes to be in close contact with the body.

Referring to FIGS. 2A and 2B, a blood pressure of an artery to be measured is PB, a pressure measured by the first pressure sensor portion 110 is P1, a pressure measured by the second pressure sensor portion 120 is P2, a distance from the upper portion of the hard structure layer 160 to the upper portion of the second pressure sensor portion 120 is A, and a distance from the upper portion of the hard structure layer 160 to the artery is B. In this case, the distance A is a predetermined constant value, P1 and P2 are pressures measured by the sensor module 100, and the distance B and the blood pressure PB are values obtained by calculation. The distance B is a distance from the upper portion of the hard structure layer 160 to a blood vessel inside the wrist, and thus, the distance B is a value measured differently depending on persons.

A pressure measured by the sensor module 100 is inversely proportional to a distance between the blood vessel and the second pressure sensor portion 120 and a distance between the blood vessel and the hard structure layer 160. Therefore, the distance B from the hard structure layer 160 to an artery may be obtained by using Equation 1 below.

$${P}_{1} ∝ \frac{{P}_{B}}{B} , \qquad Equation 1$$

$${P}_{2} ∝ \frac{{P}_{B}}{A + B} ,$$

$$\frac{{P}_{1}}{{P}_{2}} = \frac{A + B}{B} = 1 + \frac{A}{B} ,$$

$$B = \frac{{P}_{2}}{{P}_{1} - {P}_{2}} A$$

When the distance B from the upper portion of the hard structure layer 160 to the artery is obtained, the blood pressure PB may also be obtained by using Equation 2 below.

P_{B}∝B×P₁ or P_{B}∝(A+B)λP₂ Equation 2

Meanwhile, FIG. 2C is a graph illustrating pressures P1 and P2 measured by the sensor module 100 according to an embodiment of the present disclosure.

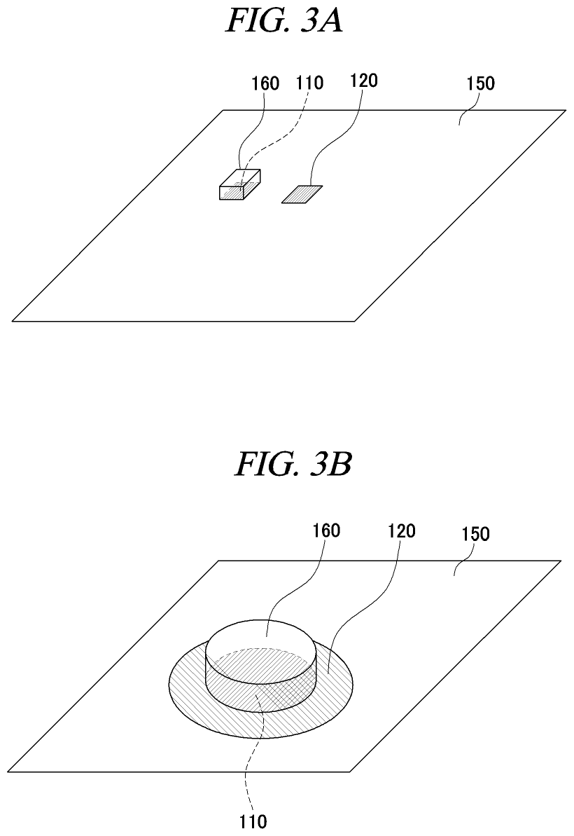

FIGS. 3A to 3C are perspective views illustrating a configuration of the sensor module according to an embodiment of the present disclosure.

The first pressure sensor portion 110 and the second pressure sensor portion 120 are disposed adjacent to each other, and the hard structure layer 160 is located on the first pressure sensor portion 110. As illustrated in FIG. 3A, the first pressure sensor portion 110 and the second pressure sensor portion 120 are located at a reference surface of the base portion 150, and the hard structure layer 160 is placed on the upper portion of the first pressure sensor portion 110, and thus, when the sensor module 100 is attached to the wrist to measure a blood pressure, the first pressure sensor portion 110 may measure a pressure at a position closer to the blood vessel.

In addition, as illustrated in FIG. 3B, the first pressure sensor portion 110 is located at the center of the base portion 150, and the second pressure sensor portion 120 is located on the reference surface of the base portion 150 in a surrounding form of surrounding the first pressure sensor portion 110. In addition, the hard structure layer 160 may be located at the upper portion of the first pressure sensor portion 110. At this time, as illustrated, the second pressure sensor portion 120 may be formed in a circular band shape surrounding the first pressure sensor portion 110. However, the second pressure sensor portion 120 is not limited to a circular shape and may also be formed in another shape.

In addition, as illustrated in FIG. 3C, the first pressure sensor portion 110 and the second pressure sensor portion 120 each include a plurality of sensors arranged in an array, the first pressure sensor portion 110 and the second pressure sensor portion 120 may be located at a reference surface of the base portion 150. The hard structure layer 160 may be located at upper portions of the plurality of first pressure sensors 110. In this case, an arrangement shape of the array of FIG. 3C may also be applied to the embodiment of FIG. 3B. That is, the first pressure sensor portion 110 and the second pressure sensor portion 120 of FIG. 3B may each include a plurality of sensors arranged in an array form.

The first pressure sensor portion 110 and the second pressure sensor portion 120 each measure pressures by using a plurality of sensors, the pressures sensed by the sensors included in each pressure sensor portion 110, and thus, when calculating a blood pressure, a maximum value, a minimum value, the most frequent value, or an average value among the pressures detected by the sensors included in the first and second pressure sensors 110 and 120 is respectively specified as the first pressure and the second pressure

An arrangement of the first and second pressure sensors 110 and 120 is not limited to the above embodiment and may be arranged in various forms.

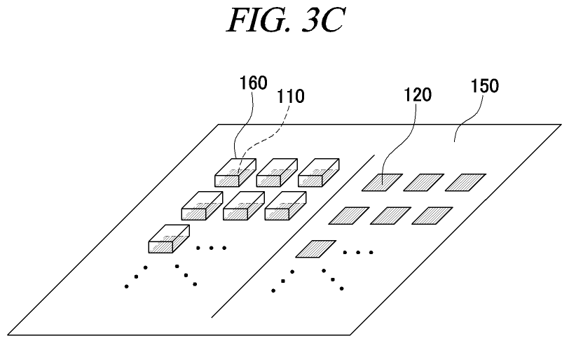

FIG. 4 is a cross-sectional view illustrating a configuration of a wrist-worn portable blood pressure measuring device according to an embodiment of the present disclosure.

A wrist-worn portable blood pressure measuring device 10 includes a sensor module 100, a main body unit 200, and a wrist strap 300. The main body unit 200 is connected to the center of the wrist strap 300 and is configured to be worn on a user's wrist. A display unit 230 is provided on an outer surface of the wrist strap 300 such that a user may easily read the displayed information.

As described above, the portable blood pressure measuring device 10 using a pressure sensor may be provided in the form of a wearable device worn on a user's wrist or so on and may be configured in various forms such as a wrist-watch, a smart band or a bracelet to be worn on the wrist.

A power supply unit 210 may be configured as a built-in type in the main body unit 200 or the wrist strap 300 or may be configured as a separate replaceable battery.

A portion A illustrated in FIG. 4 is an artery in the wrist and is a location to be considered to measure a blood pressure. The sensor module 100 is formed in the wrist strap 300 at a position close to the artery when a user wears the portable blood pressure measuring device.

The sensor module 100 may be coupled to the wrist strap 300 or the main body unit 200. The drawing illustrates that the sensor module 100 is coupled to one end of the wrist strap 300. In another embodiment, the sensor module 100 may be coupled to a lower surface of the main body unit 200.

When a user wears the portable blood pressure measuring device in the wrist strap 300, the sensor module 100 may be separated from the main body unit 200 so as to be formed at a position close to the artery. The first pressure sensor portion 110 and the second pressure sensor portion 120 are located at the reference surface of the base portion 150, and the hard structure layer 160 may be located at the upper portion of the first pressure sensor portion 110. Accordingly, there may be a difference in distance from the upper portion of the hard structure layer 160 and the upper portion of the second pressure sensor portion 120 to the artery, and the sensor module 100 may measure a blood pressure by using the difference in distance.

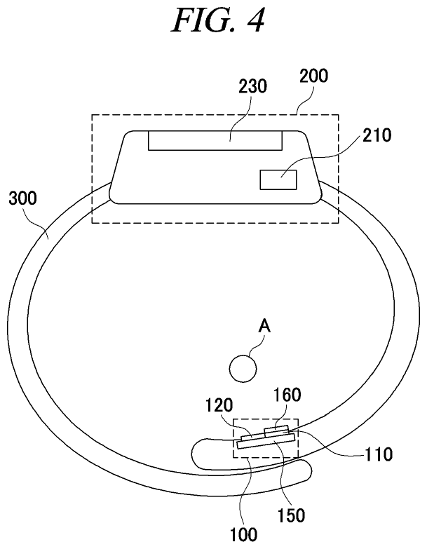

FIG. 5 is a block diagram illustrating the main body unit 200 of the portable blood pressure measuring device according to an embodiment of the present disclosure.

The main body unit 200 may include a power supply unit 210, a controller 220, a display unit 230, and a communication module 240.

The power supply unit 210 supplies power to the sensor module 100 and the main body unit 200. For example, the power supply unit 210 may supply power to the sensor module 100 at the time of measuring a blood pressure and may stop supply of the power at other times.

The controller 220 controls the sensor module 100 to measure a blood pressure through the communication module 240 when a blood pressure is required to be measure and displays the measured information on the display unit 230. For example, a blood pressure is measured in units of time, and the measured information is displayed on the display unit 230, and when a blood pressure out of a preset range is measured, an alarm occurs, or information may be transferred to a predetermined user by using the communication module 240. When the alarm occurs, a user may immediately check a high blood pressure state or a low blood pressure state.

The display unit 230 may include display monitors of various types, such as a liquid crystal display, a reflective display, and an organic light emitting diode (OLED) display. The display unit 230 may display a blood pressure calculated by the controller 220 or other types of information.

The communication module 240 may communicate with the communication module 140 of the sensor module 100 and various external devices (servers or terminals) in a communication format set respectively to transmit and receive data.

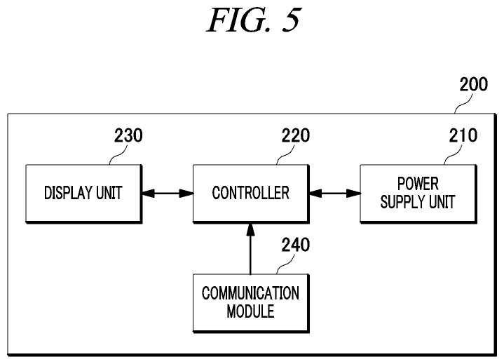

FIG. 6 is a flowchart illustrating a blood pressure measuring method using a pressure sensor, according to an embodiment of the present disclosure.

First, the first pressure sensor portion 110 and the second pressure sensor portion 120 measure pressures at each position (S110). The measured pressures are relative blood pressures that change in inverse proportion to a distance from an artery.

The controller 130 receives the first pressure P1 and the second pressure P2 measured by the first pressure sensor portion 110 and the second pressure sensor portion 120 and calculates the distance B from the hard structure layer 160 to the artery by using the first pressure P1 and the second pressure P2 (S120).

In this case, the distance B is calculated by using Equation 1 described above.

The controller 130 calculates an actual blood pressure PB by using the first pressure P1 and the second pressure P2 and the distance B from the hard structure layer 160 to the artery (S130).

In this case, the blood pressure is calculated by using Equation 2 described above.

In order to measure a diastolic blood pressure and a systolic blood pressure, a step of measuring the blood pressures a plurality of times may be further performed (S140). For example, after the blood pressures are measured at an interval of 10 ms for 10 seconds, an average of upper ten measured values may be determined as the systolic blood pressure, and an average of lower ten measured values may be determined as the diastolic blood pressure. In order to measure the blood pressures more accurately, the measurement interval may be set to 1 ms.

The controller 220 displays information including the diastolic blood pressure and the systolic blood pressure on the display unit 230 (S150). In this case, when the blood pressures are out of a preset normal range, an alarm to notify of high or low blood pressure may occur or an alarm message may be transmitted to another device such as a preset mobile phone.

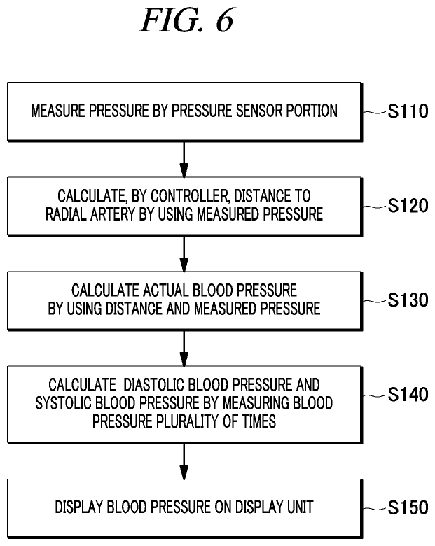

FIG. 7 is a graph illustrating a change in arterial blood pressure over time, which may be referred to when determining a measurement interval for measuring the diastolic blood pressure and the systolic blood pressure.

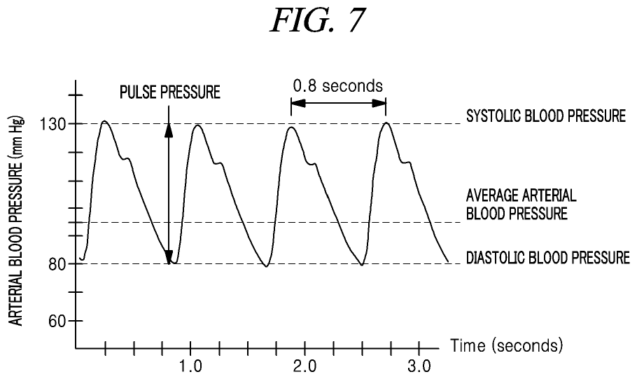

FIGS. 8 to 10 are views illustrating a stacked structure and a detailed configuration of a sensor module for measuring a pressure according to an embodiment of the present disclosure.

As illustrated, a sensor module 400 for measuring a pressure may include a first electrode 410, a second electrode 420, and a dielectric layer 430. In addition, the sensor module 400 for measuring a pressure may be used as the first and second pressure sensors 110 and 120 of the sensor module 100 for measuring the blood pressures described above.

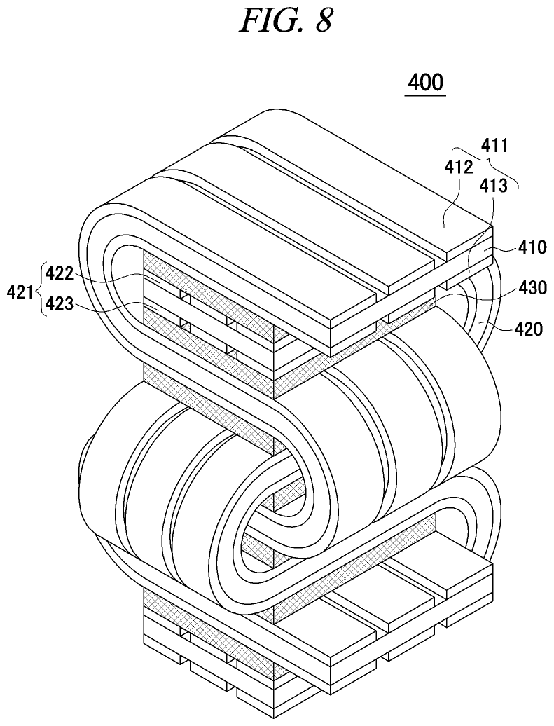

Referring to FIG. 9, N electrode wires 411, which are included in the first electrode 410 and are separated from each other, may be stacked in parallel to each other on upper and lower surfaces of a base layer. For example, the first electrode 410 may include upper surface electrode wires 412 including three wires separated by a predetermined distance and arranged on an upper surface of a base layer, and lower surface electrode wires 413 including three wires separated by a predetermined distance downward from the electrode wires 412 arranged on the upper surface of the base layer, but the number of electrode wires 411 is not limited thereto.

In addition, electrode wires 421 may be formed in the second electrode 420 in the same shape as the first electrode 410. In other words, the second electrode 420 may include N electrode wires 421 which are separated from each other and are stacked in parallel to each other on the upper surface and the lower surface of the base layer. For example, the second electrode 420 may include upper surface electrode wires 422 including three wires separated by a predetermined distance and arranged on an upper surface of a base layer, and lower surface electrode wires 423 including three wires separated by a predetermined distance downward from the electrode wires 422 arranged on the upper surface, but the number of electrode wires 421 is not limited thereto.

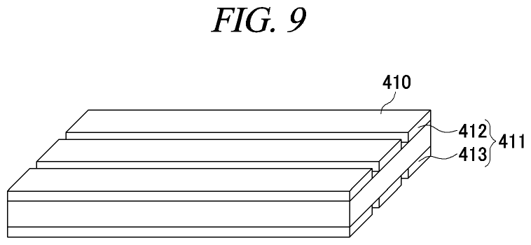

Referring to FIG. 10, inner bending of the first electrode 410 and inner bending of the second electrode 420 are repeated for each unit area, and the electrode wires 411 of the first electrode 410 may cross the electrode wires 421 of the second electrode 420, and the dielectric layer 430 may be formed between the electrode wires 411 of the first electrode 410 and the electrode wires 421 of the second electrode 420 which face each other and cross each other.

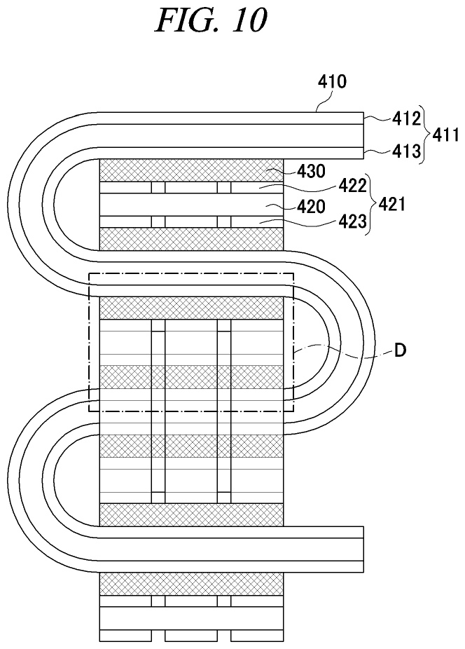

FIG. 11 is an enlarged view of D of FIG. 10. Referring to FIGS. 10 and 11, in the first electrode 410, the electrode wires 412 which have bent inner sides and are arranged on the upper surface, may face each other and may set as a first upper surface electrode wire 412a and a second upper surface electrode wire 412b facing each other, the second electrode 420 may be disposed between the first upper surface electrode wire 412a and the second upper surface electrode wire 412b, and thereby, electrode wires of the second electrode 420 may be set as a first lower surface electrode wire 423a of the second electrode 420 which faces the first upper surface electrode wire 412a and a first upper surface electrode wire 422a of the second electrode 420 which faces the second upper surface electrode wire 412b. In this case, the sensor module 400 may include a first dielectric layer 431 disposed between the first upper surface electrode wire 412a of the first electrode 410 and the first lower surface electrode wire 423a of the second electrode 420, and a second dielectric layer 432 disposed between the second upper surface electrode wire 412b of the first electrode 410 and the first upper surface electrode wire 422a of the second electrode 420.

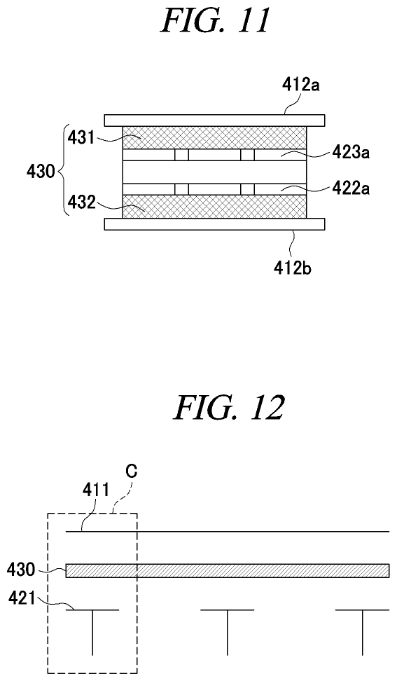

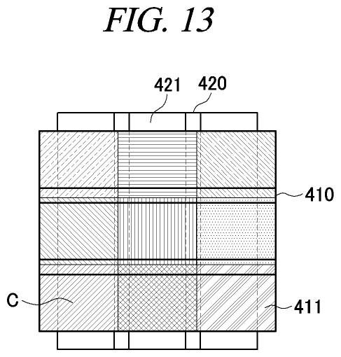

FIGS. 12 and 13 are views illustrating that a capacitor C is formed in the sensor module 400 for measuring a pressure according to an embodiment of the present disclosure.

The sensor module 400 may include N*N capacitors C formed in each unit area and measure change values of the capacitors C to detect a pressure. Referring to FIG. 12, the capacitor C may include the electrode wire 411 of the first electrode 410, the electrode wire 421 of the second electrode 420, and the dielectric layer 430 between the electrode wire 411 of the first electrode 410 and the electrode wire 421 of the second electrode 420. For example, referring to FIG. 13, when the electrode wires 411 of the first electrode 410 are separated into three wires and the electrode wires 421 of the second electrode 420 are separated into three wires, nine capacitors C may be formed at intersections of the electrode wires 411 of the first electrode 410 and the electrode wires 421 of the second electrode 420.

In addition, when the first electrode 410 and the second electrode 420 are each bent at M locations, the sensor module 400 has 2M+1 unit areas, and in this case, the sensor module 400 may have (N*N)*(2M+1) capacitors C. For example, as illustrated in FIG. 10, when the first electrode 410 and the second electrode 420 are each bent at three locations and three electrode wires 411 and 421 are separately arranged on the upper and lower surfaces, the sensor module 400 may have seven unit areas, and 63 capacitors C may be formed therein.

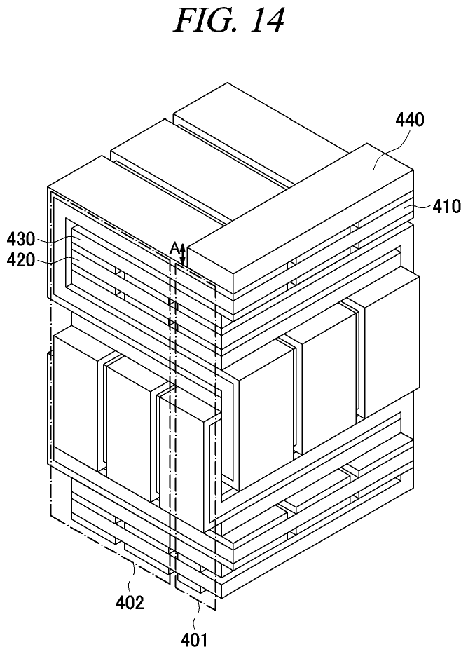

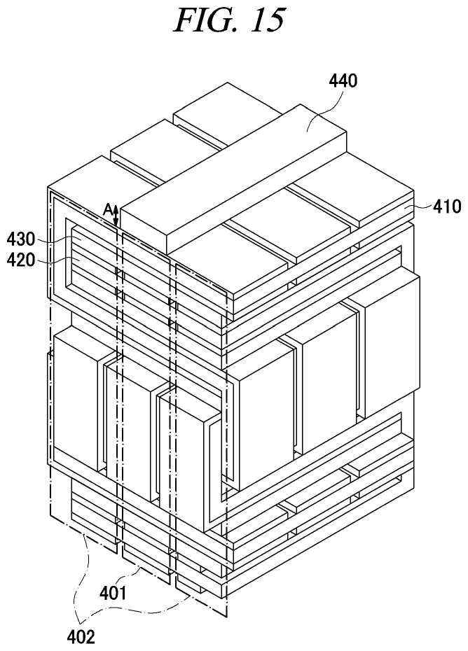

FIGS. 14 to 15 are perspective views illustrating a configuration of a sensor module for measuring a pressure according to an embodiment of the present disclosure.

As illustrated, an upper surface of the sensor module 400 may include a hard structure layer 440 coupled thereto to cover a predetermined area of the sensor module 400, and accordingly, a step difference of a first distance A may be formed between an upper portion of the hard structure layer 400 and an upper portion of the sensor module 400 not covered by the hard structure layer 440.

As illustrated in FIG. 14, the hard structure layer 440 may have a predetermined area on one side of an upper portion of the first electrode 410, and at least one or more capacitors C may be provided under the hard structure layer 440. In addition, as illustrated in FIG. 15, the hard structure layer 440 may be formed in the center of an upper portion of the first electrode 410 to have a predetermined area, and at least one capacitor C may be formed under the hard structure layer 440. Although FIGS. 14 and 15 illustrate that the hard structure layer 440 is formed in a rectangular parallelepiped shape, the hard structure layer 440 is not limited thereto and may be formed in other shapes.

A controller may control an operation of a first pressure sensor 401 located in a region covered with the hard structure layer 440 and an operation of a second pressure sensor 402 located in a region not covered with the hard structure layer 440 and may measure a blood pressure of a blood pressure measurement target blood vessel based on a first pressure detected by the first pressure sensor 401 and a second pressure detected by the second pressure sensor 402. In this case, the controller estimates a second distance indicating a distance from an upper portion of the hard structure layer 440 to the blood pressure measurement target blood vessel based on the first pressure, the second pressure, and the first distance A and may calculate a blood pressure value of a target blood vessel based on a value obtained by multiplying the first pressure by a second distance or a value obtained by multiplying the sum of the first distance A and the second distance by the second pressure.

According to the above-described means for solving the problems of the present disclosure, a blood pressure measuring device using a pressure sensor according to an embodiment of the present disclosure may measure a blood pressure only with the pressure sensor without a procedure of applying a pressure to a target point by using an air pump in a blood pressure measurement process, and thus, a user measures a blood pressure conveniently at any time.

In addition, the blood pressure measuring device using a pressure sensor according to an embodiment of the present disclosure does not use an air pump that applies a pressure to measure a blood pressure. Accordingly, a manufacturing process may be simplified, costs may be reduced, and the blood pressure measuring device may be manufactured in a small size, and thus, a user's portability and convenience may be increased.

In addition, a sensor module for measuring a blood pressure according to an embodiment of the present disclosure may be attached to a target point in the form of various types of wearable devices, such as clothing, in addition to a wrist-worn form to measure a blood pressure.

An embodiment of the present disclosure may also be implemented in the form of a recording medium including instructions executable by a computer, such as a program module executed by the computer. Computer-readable media may be any available media that may be accessed by a computer and include both volatile and nonvolatile media and removable and non-removable media. In addition, the computer-readable media may include all computer storage media. The computer storage media includes both volatile and nonvolatile media and removable and non-removable media implemented by any method or technology of storing information, such as a computer readable instruction, a data structure, a program module, and other data.

Although the method and system according to the present disclosure are described with reference to specific embodiments, some or all of their components or operations may be implemented by using a computer system having a general-purpose hardware architecture.

The above descriptions on the present disclosure are for illustration, and those skilled in the art to which the present disclosure pertains may understand that the descriptions may be easily modified into other specific forms without changing the technical idea or essential features of the present disclosure. Therefore, it should be understood that the embodiments described above are illustrative in all respects and not restrictive. For example, each component described as a single type may be implemented in a dispersed form, and likewise components described as distributed may be implemented in a combined form.

The scope of the present disclosure is indicated by the following claims rather than the above detailed description, and all changes or modifications derived from the meaning and scope of the claims and their equivalents should be interpreted as being included in the scope of the present disclosure.

What is claimed is:

1. A sensor module for measuring a blood pressure, comprising:
a base portion;
a first pressure sensor portion and a second pressure sensor portion coupled to the base portion and arranged adjacent to each other; and
a hard structure layer coupled to an upper portion of the first pressure sensor portion,
wherein a step difference of a first distance is formed between an upper portion of the hard structure layer and the upper portion of the second pressure sensor portion.

2. The sensor module of claim 1, further comprising:
a controller configured to control operations of the first pressure sensor portion and the second pressure sensor portion and configured to measure a blood pressure of a blood pressure measurement target blood vessel based on a first pressure detected by the first pressure sensor portion and a second pressure detected by the second pressure sensor portion,
wherein the controller estimates a second distance indicating a distance from the upper portion of the hard structure layer to the blood pressure measurement target blood vessel based on the first pressure, the second pressure, and the first distance, and calculates a blood pressure of the blood pressure measurement target blood vessel based on a value obtained by multiplying the second distance by the first pressure or a value obtained by multiplying a sum of the first distance and the second distance by the second pressure.

3. The sensor module of claim 1, wherein
each of the first pressure sensor portion and the second pressure sensor portion is one of a strain gauge-based semiconductor thin film sensor, a capacitive thin film sensor for detecting a change in capacitance according to a pressure, and a piezoresistive sensor using a piezo resistance effect.

4. The sensor module of claim 1, wherein
the first pressure sensor portion is located at a central portion of the base portion, and the second pressure sensor portion is coupled to the first pressure sensor portion in a form surrounding the first pressure sensor portion.

5. The sensor module of claim 1, wherein
each of the first pressure sensor portion and the second pressure sensor portion includes a plurality of sensors arranged in an array.

6. The sensor module of claim 2, wherein
each of the first pressure sensor portion and the second pressure sensor portion includes a plurality of sensors arranged in an array, and
the controller sets a maximum value, a minimum value, a most frequent value, or an average value among pressures detected by sensors included in the first pressure sensor portion as the first pressure, and sets a maximum value, a minimum value, a most frequent value, or an average value among pressures detected by sensors included in the second pressure sensor portion as the second pressure.

7. A wrist-worn portable blood pressure measuring device comprising:
a main body unit including a display unit for displaying blood pressure information, a power supply unit, and a controller;
a wrist strap coupled to the main body unit; and
a sensor module coupled to one of the main body unit and the wrist strap to measure a blood pressure,
wherein the sensor module includes a first pressure sensor portion and a second pressure sensor portion which are coupled to a base portion and arranged adjacent to each other, and a hard structure layer coupled to an upper portion of the first pressure sensor portion, and
a step difference of a first distance is formed between an upper portion of the hard structure layer and an upper portion of the second pressure sensor portion.

8. The portable blood pressure measuring device of claim 7, wherein
the controller estimates a second distance indicating a distance from the upper portion of the hard structure layer to a blood pressure measurement target blood vessel based on a first pressure, a second pressure, and the first distance, and calculates a blood pressure of the blood pressure measurement target blood vessel based on a value obtained by multiplying a second distance by the first pressure or a value obtained by multiplying a sum of the first distance and the second distance by the second pressure, and outputs the calculated blood pressure through the display unit.

9. The portable blood pressure measuring device of claim 7, wherein
each of the first pressure sensor portion and the second pressure sensor portion is one of a strain gauge-based semiconductor thin film sensor, a capacitive thin film sensor for detecting a change in capacitance according to a pressure, and a piezoresistive sensor using a piezo resistance effect.

10. The portable blood pressure measuring device of claim 7, wherein
the first pressure sensor portion is located at a central portion of the base portion, and the second pressure sensor portion is coupled to the first pressure sensor portion in a form surrounding the first pressure sensor portion.

11. The portable blood pressure measuring device of claim 7, wherein
each of the first pressure sensor portion and the second pressure sensor portion includes a plurality of sensors arranged in an array, and
the controller sets a maximum value, a minimum value, a most frequent value, or an average value among pressures detected by sensors included in the first pressure sensor portion as the first pressure, and sets a maximum value, a minimum value, a most frequent value, or an average value among pressures detected by sensors included in the second pressure sensor portion as the second pressure.

12. The portable blood pressure measuring device of claim 7, further comprising:
a communication module that performs data communication,
wherein the controller transmits the measured blood pressure information to an external device through the communication module.

13. A sensor module for measuring a pressure, comprising:
a first electrode including N separated electrode wires stacked in parallel to each other on an upper surface and a lower surface of a base layer and having inner bending repeated for each unit area;
a second electrode which includes N separated electrode wires stacked in parallel to each other on an upper surface and a lower surface of a base layer and has inner bending repeated for each unit area and in which the electrode wires of the first electrode cross the electrode wires of the second electrode; and
a plurality of dielectric layers inserted between the electrode wires of the first electrode and the electrode wires of the second electrode that arranged to face and cross each other.

14. The sensor module of claim 13, wherein
N*N capacitors are formed in each unit area.

15. The sensor module of claim 13, wherein
the electrode wires arranged on the upper surface of the first electrode face each other according to the inner bending, and the electrode wires are set as first upper surface electrode wires and second upper surface electrode wires which face each other,
the second electrode is disposed between the first upper surface electrode wires and the second upper surface electrode wires, and the electrode wires of the second electrode are set as a first lower surface electrode wire of the second electrode facing the first upper surface electrode wires and first upper surface electrode wires of the second electrode facing the second upper surface electrode wires,
a first dielectric layer is disposed between the first upper surface electrode wires of the first electrode and the first lower surface electrode wires of the second electrode, and
a second dielectric layer is disposed between the second upper surface electrode wires of the first electrode and the first upper surface electrode wires of the second electrode.

16. The sensor module of claim 13, further comprising:
a hard structure layer coupled to an upper surface of the sensor module to cover a predetermined area of the sensor module,
wherein a step difference of a first distance is formed between an upper portion of the hard structure layer and an upper portion of the sensor module not covered by the hard structure layer.

17. The sensor module of claim 13, further comprising:
a controller configured to control an operation of a first pressure sensor portion located at a region covered with the hard structure layer and an operation of a second pressure sensor portion located at a region not covered with the hard structure layer and configured to measure a blood pressure of a blood pressure measurement target blood vessel based on a first pressure detected by the first pressure sensor portion and a second pressure detected by the second pressure sensor portion,
wherein the controller estimates a second distance indicating a distance from an upper portion of the hard structure layer to the blood pressure measurement target blood vessel based on the first pressure, the second pressure, and a first distance, and calculates a blood pressure of the blood pressure measurement target blood vessel based on a value obtained by multiplying the second distance by the first pressure or a value obtained by multiplying a sum of the first distance and the second distance by the second pressure.
